(19) United States
(12) Patent Application Publication
(10) Pub. No.: US 2009/0263906 A1
(43) Pub. Date: Oct. 22, 2009

(54) METHOD OF ANTIOXIDATIVE FUNCTIONAL ESTIMATION USING ANIMAL MODEL

(75) Inventors: Il-Yun JEONG, Jeongeup-si (KR); Yong Dae PARK, Jeongeup-si (KR); Chang-Hyun JIN, Jeongeup-si (KR); Dae-Seong CHOI, Jeongeup-si (KR); Myung-Woo BYUN, Jeongeup-si (KR); Hyo-Jung LEE, Cheongju-si (KR)

Correspondence Address: GREENLEE WINNER AND SULLIVAN P C, 4875 PEARL EAST CIRCLE, SUITE 200, BOULDER, CO, 80301, US

(73) Assignee: KOREA ATOMIC ENERGY RESEARCH INSTITUTE, Daejeon (KR)

(21) Appl. No.: 12/252,917

(22) Filed: Oct. 16, 2008

(30) Foreign Application Priority Data
Apr. 17, 2008 (KR) KR10-2008-0035724

(51) Int. Cl. G01N 33/92

(52) U.S. Cl. 436/71

(57) ABSTRACT

The present invention relates to a method of antioxidative functional estimation using an animal model, more precisely a method of antioxidative functional estimation using mice having oxidative damage caused by reactive oxygen species induced by irradiation and having lipid hydroperoxide secreted in the urine which might be index for quantitative and qualitative analysis for antioxidative functional estimation. The method of antioxidative functional estimation of the present invention can be effectively used for the screening of a novel anti-oxidant agent or antioxidative functional health food to regulate the production of lipid hydroperoxide.

### TECHNICAL FIELD

The present invention relates to a method of antioxidative functional estimation using an animal model.

### BACKGROUND ART

According to the disclosure that reactive oxygen species and peroxides are one of direct reasons of disease and work as oxidation induction target factors (Pryor, A. et al., Free Radic. Biol. Med., 8, 541-543, 1990), studies have been actively going on in order to find out an oxidation induction target regulator at home and overseas. Even after all the efforts to develop a novel anti-oxidative material as an effort to prevent disease and aging and further to realize and commercialize the material (Hyra, Y. et al., Plenum press, pp. 49-66), pre-clinical evaluation technique for the safe application in human has not been satisfactorily advanced that much.

The conventional evaluation method of antioxidative effect comprises in-vitro, ex-vivo, in-vivo, and human tests. The most representative in-vitro tests are lipid peroxidation inhibition assay, total antioxidant activity assay DPPH (a kind of free radical) scavenging activity test (Gutteridge, M. et al., Anal. Biochem., 91, 250, 1978). These days, chemiluminiscence assay directly measuring radicals generated, electron spin resonance (ESR) spin-trapping method indirectly measuring radicals and deoxyribose assay have been developed. Particularly, to test the activity to inhibit damages on DNA, protein and lipid in human body, tissues or cells are extracted and analyzed by using 8-oxoguanosine assay, carbonyl-containing product measurement and oxidized LDL production inhibition test. Recent overseas reports on antioxidation in human body are largely focused on single component analysis using anti-oxidation biomarkers such as antioxidant enzyme activity (SOD) in erythrocytes, GDH-Px, catalase, lipid hydroperoxide level (MDA), DNA damage level (lymphocyte DNA damage level measured by Comet assay), 8-hydroxy-2′-deoxyguanisine level in urine and anti-oxidant vitamin (vitamin E, carotenoids, vitamin C] level in serum.

The field of anti-oxidation related study is so wide and there has been no acknowledged theory on oxidation mechanism so far. Besides, interpretations on the results of anti-oxidation estimation are varied. Therefore, after all the studies to establish a proper method for estimating anti-oxidative activity (Mazza, G. et al., AOCS Press., 1997, pp. 119-140), it is still very difficult not only to evaluate the anti-oxidative effect in normal healthy human by taking health functional food and to track down biomarkers but also to estimate and evaluate the anti-oxidative effect because of so many variants such as oxidative stress factors like drinking, smoking and exercise and homeostatic tendency controlled by in vivo anti-oxidative mechanism.

The present inventors have studied on the effect of radiation energy on organic molecules in vivo. In the meantime, the present inventors generated a mouse model in which lipid hydroperoxide is secreted in urine by oxidative damage caused by the attack of reactive oxygen species induced by irradiation. At last, the present inventors completed this invention by confirming that an anti-oxidant agent or anti-oxidative health food that can control the production of lipid hydroperoxide can be screened by using the mouse model.

### [Disclosure]

### [Technical Problem]

It is an object of the present invention to provide an animal model designed to secrete lipid hydroperoxide in urine by irradiation and a method for screening a lipid hydroperoxide regulator using an animal model as well as a method for antioxidative functional estimation.

### [Technical Solution]

To achieve the above object, the present invention provides a screening method of a lipid hydroperoxide regulator using an animal model in which lipid hydroperoxide generated by oxidative damage resulted by the attack of reactive oxygen species (ROS) induced by irradiation is secreted into urine.

The present invention also provides a screening method of an anti-oxidant agent using the said animal model.

### [Advantageous Effect]

In this invention, an animal model was generated by irradiating to induce anti-oxidative stress in order for lipid hydroperoxide to be secreted in urine. This animal model can be effectively used for screening anti-oxidative functional food and medicine for the prevention of disease and aging by analyzing and regulating the lipid hydroperoxide.

### DESCRIPTION OF DRAWINGS

The application of the preferred embodiments of the present invention is best understood with reference to the accompanying drawings, wherein:

FIG. 1 is a diagram illustrating the extraction of white lotus used as an anti-oxidant agent and purification process of the sample of the present invention.

FIG. 2 is a diagram illustrating the process of taking urine by using metabolic cage after irradiation on the mouse.

FIG. 3 is a graph illustrating the changes of phenylhydrazone in the non-irradiated group, the irradiated group and the group irradiated and administered with the fraction of white lotus leaf extract as an anti-oxidant agent.

FIG. 4 is a graph illustrating the detection time and molecular weight of phenylhydrazone detected by gas chromatography/Mass Selective Detector (GC/MSD).

FIG. 5 is a set of graphs illustrating the phenylhydrazone derivative originated from isobutylaldehyde detected by GC/MSD.

FIG. 6 is a set of graphs illustrating the phenylhydrazone derivative originated from 2-methylbutylaldehyde detected by GC/MSD.

FIG. 7 is a set of graphs illustrating the phenylhydrazone derivative originated from isovaleraldehyde detected by GC/MSD.

FIG. 8 is a set of graphs illustrating the phenylhydrazone derivative originated from valeraldehyde detected by GC/MSD.

FIG. 9 is a diagram illustrating the in-vitro synthesis of phenylhydrazone to confirm whether the detected phenylhydrazone derivative is originated from the corresponding aldehyde.

### BEST MODE

Hereinafter, the present invention is described in detail.

In this invention, the term “adaptation” indicates the process of gradual adaption or being used to a new environment after being transferred.

The present invention provides a screening method of a lipid hydroperoxide regulator comprising the following steps:

1) irradiating the experimental group animals treated with candidate substances and the control group animals;

2) collecting urines from the animals of step 1);

3) performing quantitative and qualitative analysis of lipid hydroperoxide in urines collected in step 2) and comparing the levels between the experimental group and the control group; and

4) selecting a candidate substance that made changes in components or quantity of lipid hydroperoxide by comparing the results of the control group and the experimental group of step 3).

In this method, the candidate substance of step 1) is selected from the group consisting of peptide, protein, non-peptide compound, synthetic compound, fermented product, cell extract, plant extract, animal tissue extract and blood plasma, but not always limited thereto and any substance that is edible and presumed to have anti-oxidative effect can be accepted.

In this method, the animal of step 1) is selected from the group consisting of mouse, rat, pig and monkey, but mouse or rat is preferred, but not always limited thereto and any mammal can be used. The mouse herein is Balb.c, ICR or C57BL/6j, and the rat herein is preferably SD or Wistar-ST, but not always limited thereto.

In this method, the radiation of step 2) is selected from the group consisting of gamma ray, electron beam, X ray, ion beam and UV, and gamma ray is more preferred considering that gamma ray has stronger permeability than any other radiations, but not always limited thereto. The gamma ray herein is preferably emitted from an isotope selected from the group consisting of Co-60, Kr-85, Sr-90 and Cs-137, but not always limited thereto.

Irradiation is preferably performed to a whole animal body for 3-8 minutes with the total absorbed dose of 2-4 Gy, but not always limited thereto. If the total absorbed dose is less than 2 Gy, lipid hydroperoxide is not properly secreted. If the total absorbed dose is more than 4 Gy, it is not good because it approaches to the lethal dose of 6 Gy.

In this method, the urine collection of step 2) is performed preferably at −15° C. to 10 by using metabolic cage, but not always limited thereto.

In this method, the analysis of lipid hydroperoxide of step 3) is preferably performed as follows; phenylhydrazine is added to aldehyde detected in urine to synthesize phenylhydrazone derivative, followed by analysis thereof, but not always limited thereto.

The phenylhydrazine herein is selected from the group consisting of 2,4-dinitrophenylhydrazine, 4-chlorophenylhydrazine and 2,4-dichlorophenylhydrazine, but not always limited thereto.

It is more preferred to add a step of adapting test animals before step 1). Adaptation is preferably performed at 20-30° C. for 2-10 days, and is more preferably performed at 21-25° C. for 4-7 days, but not always limited thereto.

5 week old female Balb.c mice were adapted in a cage at 22±1° C. with 12 hour light/dark cycle for one week. Then, 0.9% NaCl/D.W was orally administered for 4 days. On the 4^{th} day, the mice were irradiated on the whole body with ¹³⁷Cs-gamma ray at 0.84 Gy/min with absorbed dose of 4 Gy to induce oxidative stress. Reactive oxygen species (ROS) generation was induced in those irradiated mice which attacked the cells, and lipid hydroperoxides produced by such oxidative damage were secreted into urine.

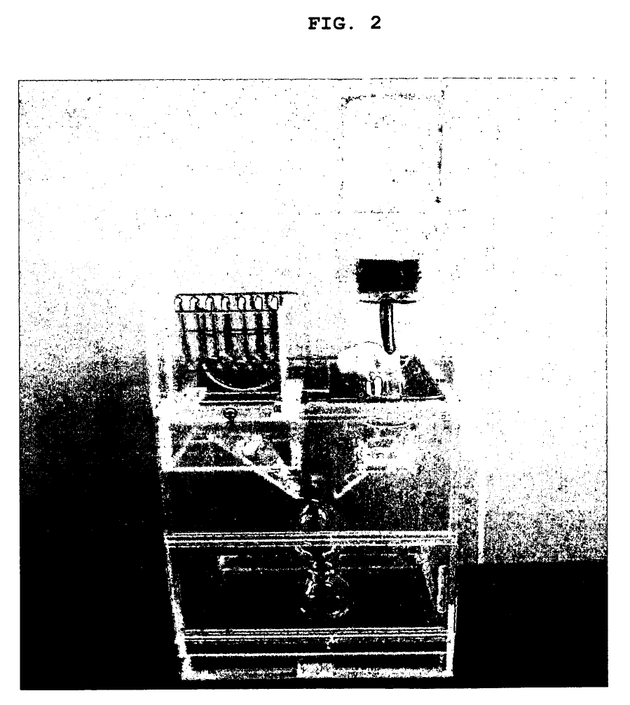

The present inventors transferred the irradiated group and non-irradiated group to metabolic cage (see FIG. 2) and provided them only water without feeds for 12 hours. Then, urine was collected and stored at −20° C. for further use.

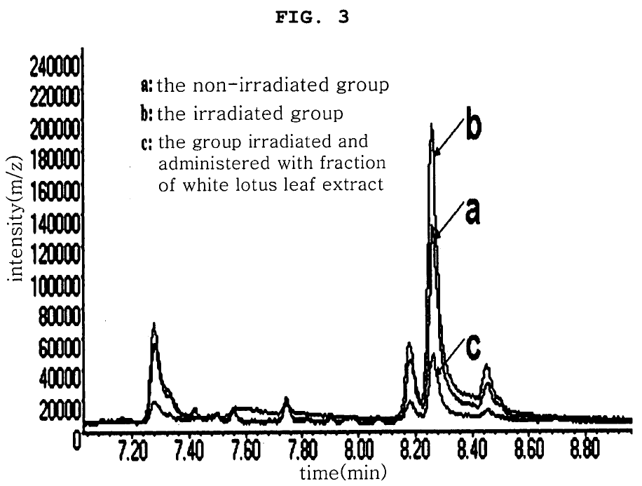

To analyze the level of aldehyde, a kind of lipid hydroperoxides, in the urine samples, lipid hydroperoxide assay (Chghetti, G. et al., Anal. Biochem., 266, 222-229, 1999) was performed by using gas chromatography/Mass Selective Detector (GC/MSD). As a result, the level of phenylhydrazone derivative was significantly increased in the irradiated group, compared with that in the non-irradiated group (see FIG. 3). This result suggests that the level of lipid hydroxide aldehyde, aldehyde, in the urine sample of the irradiated group was higher than that of the non-irradiated group.

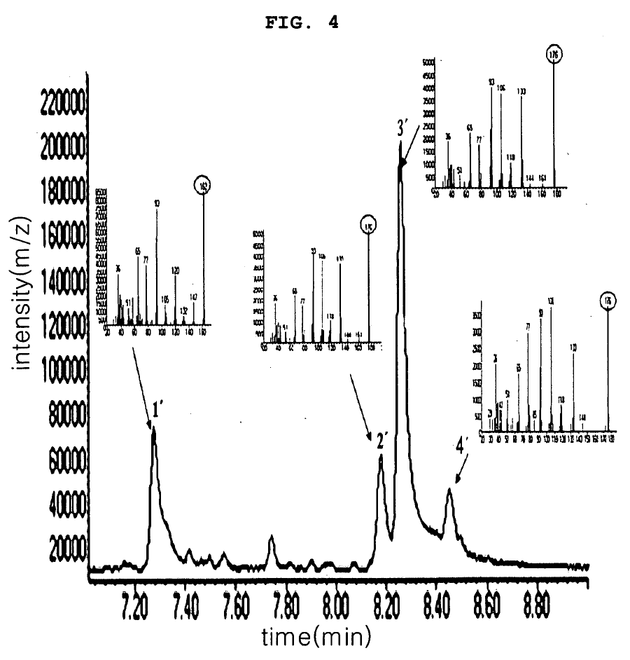

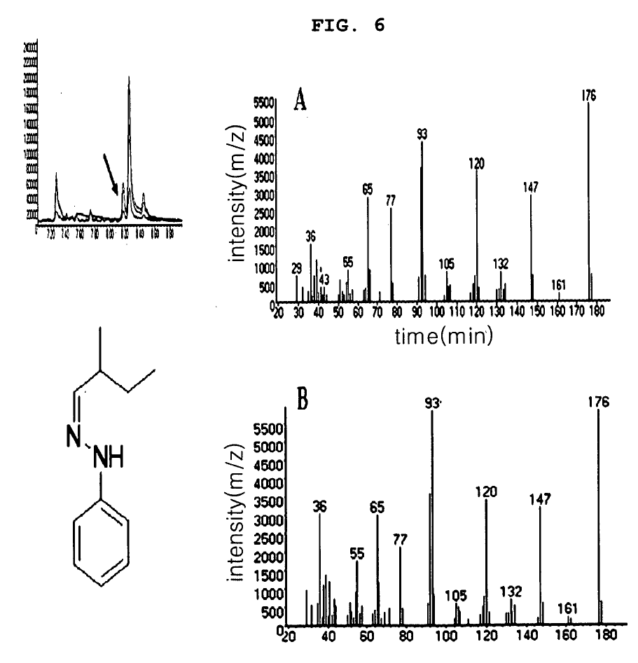

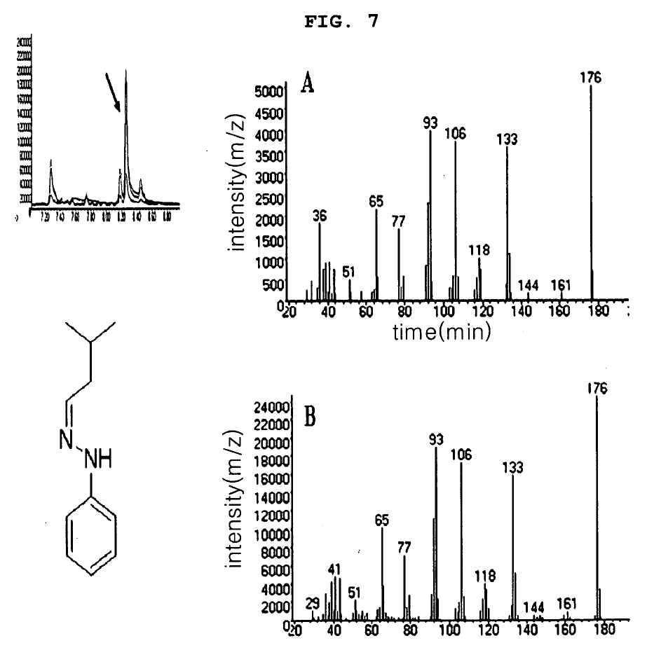

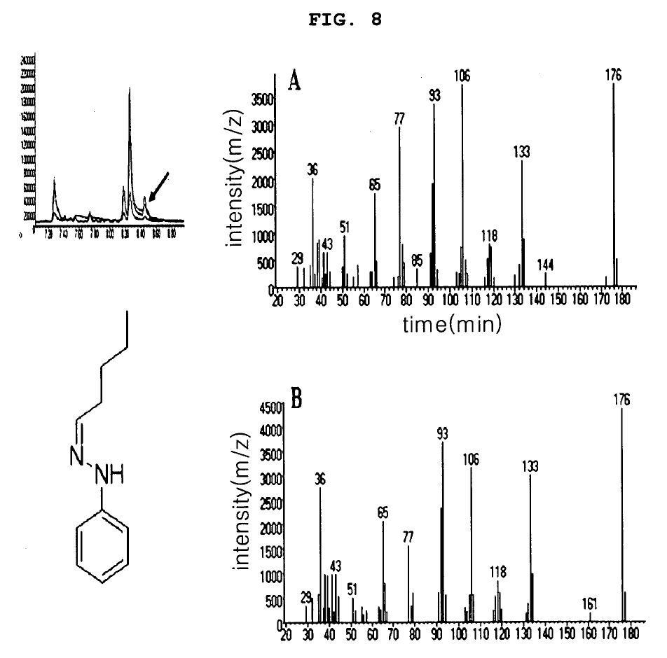

To identify the structure of phenylhydrazone derivative, the present inventors induced the reaction of aldehyde corresponding to the phenylhydrazone derivative and phenylhydrazine to compare and analyze the phenylhydrazone derivative in the urine. As a result, it was confirmed that the phenylhydrazone derivative was derived from isobutylaldehyde, 2-methylbutylaldehyde, isovaleraldehyde or valeraldehyde (see FIG. 4-FIG. 8). So, identification of lipid hydroperoxide secreted in the urine of the irradiated mouse facilitates the analysis of lipid hydroperoxide.

### [Mode for Invention]

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of White Lotus Leaf Extract

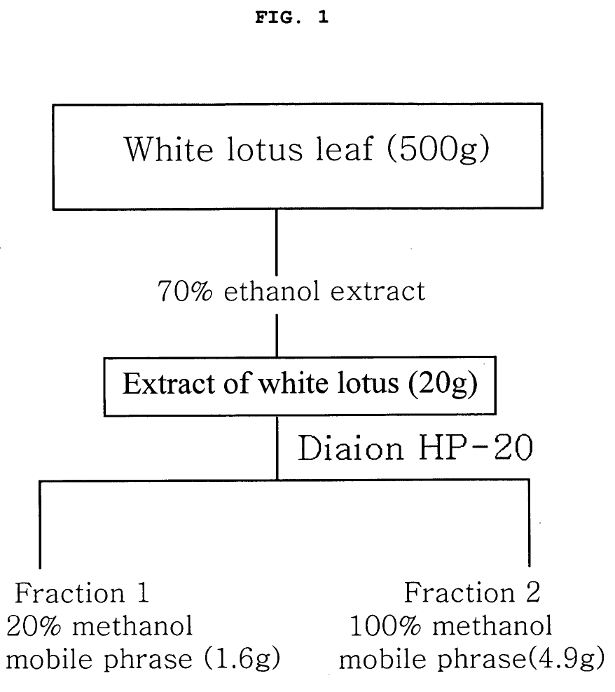

500 g of white lotus leaf collected in Jeongeup, Jeollabuk-do, Korea was dipped in 4× 70% methanol, which stood for 24 hours. Extract and solid residue were obtained by filtering the solution with a filter paper. The obtained extract was concentrated under reduced pressure to give 20 g of white lotus leaf extract. The extract was fractionated by using Diaion HP-20 column. As a result, 1.6 g (20% methanol) and 4.9 g (100% methanol) of fractions were obtained (FIG. 1). The obtained white lotus methanol fractions were tested for in vivo ROS scavenging activity.

### EXAMPLE 2

### Adaptation of Mice

24 female Balb.c mice at 5 weeks (Orientbio Inc., Korea) were adapted in a cage at 22±1° C. with 12 hour light/dark cycle for one week. Pellet feeds for test animals and water were provided freely.

### EXPERIMENTAL EXAMPLE 1

### Preparation of Animal Model

### <1-1> Irradiation and Sample Administration

The test animals prepared in Example 2 were grouped into three (control group, irradiated group, irradiated+sample administered group). The control group (n=8) was orally administered with 0.9% NaCl/D.W for 4 days. The irradiated group (n=8) was orally administered with 0.9% NaCl/D.W for 4 days and on the 4^{th} day, the animals were irradiated on the whole body at 10 pm. The irradiated+sample administered group was orally administered with white lotus leaf methanol extract (50 mg/kg body weight) obtained in Example 1 for 4 days before irradiation. Oral administration was performed at 4 pm by 0.5 ml equally. Irradiation was performed in ARTI (Advanced Radiation Technology Institute) using GammaCell-40 (Nordion, Canada) with ¹³⁷Cs gamma ray at room temperature (12±1° C.) at the dose of 0.84 Gy (absorbed dose: 4 Gy).

### <1-2> Collection of Urine Using Metabolic Cage

The mice of the irradiated group and the irradiated+sample administered group of Experimental Example <1-1> were irradiated and then transferred to metabolic cage (FIG. 2). Urine samples were collected for 12 hours. During the sampling, only water was provided without feeds. The collected urine samples were stored at −20° C. before experiments.

### EXPERIMENTAL EXAMPLE 2

### Analysis of Lipid Hydroperoxide Secreted by Irradiation

### <2-1> GC/MSD Conditions

GC/MSD analysis was performed with 5890 gas chromatography (Agilent, U.S.A) equipped with HP-5 fused silica capillary column (25 m, 0.32 mm i.d., 0.25 mm film) and 5988A mass spectrometer. Helium gas was used as moving phase and flow rate was set at 0.7 ml/min. Splitless injection mode was used. Starting temperature of the oven was 70° C., which was raised 25° C. per minute up to 175° C. From 175° C., temperature was raised 5° C. per minutes up to 200° C. Temperature was raised to 300° C., which was maintained for 10 minutes. Mass analysis was performed by using electron ionization mode and ion source temperature was set at 180° C.

### <2-2> GS/MSD Analysis

Lipid hydroperoxide assay was performed using gas chromatography/Mass Selective Detector (GC/MSD) (Chghetti, G. et al., Anal. Biochem., 266, 222-229, 1999). First, phenylhydrazine was used to detect aldehyde from the urine samples collected in Example <1-2>. Derivative reagent was prepared by adding 22 ml of phenylhydrazine to 5 ml of 2 M HCl. 2 ml of the urine sample was loaded in 50 ml screw-capped TEFE tube, to which 7.6 ml of distilled water and 0.4 ml of the derivative reagent prepared above were added, followed by shaking for 10 minutes to produce phenylhydrazone by the reaction of phenylhydrazine and aldehyde secreted in urine. Then, 10 ml of hexane was added thereto, followed by shaking for 10 minutes, and then centrifugation was performed at 3000 rpm for 10 minutes. Hexane layer was separated, followed by concentration and dissolved in chloroform, resulting in the preparation of sample for GC/MSD analysis. Aldehyde levels in the urine samples of the non-irradiated group, the irradiated group and the irradiated+white lotus extract administered group were measured under the same conditions as GC/MSD analysis conditions described in Example <2-1> (FIG. 3).

As a result, phenylhydrazone derivatives were confirmed (MW 162 at 7.21 min., MW 176 at 8.18 min., MW 176 at 8.26 min., MW 176 at 8.44 min.) (FIG. 4). Those derivatives were significantly increased in the irradiated group, compared with those in the non-irradiated group and decreased in the group treated with white lotus extract, the anti-oxidant agent.

To identify the structure of phenylhydrazone derivative, the present inventors induced the reaction of aldehyde corresponding to the phenylhydrazone derivative and phenylhydrazine to compare and analyze the phenylhydrazone derivative in the urine.

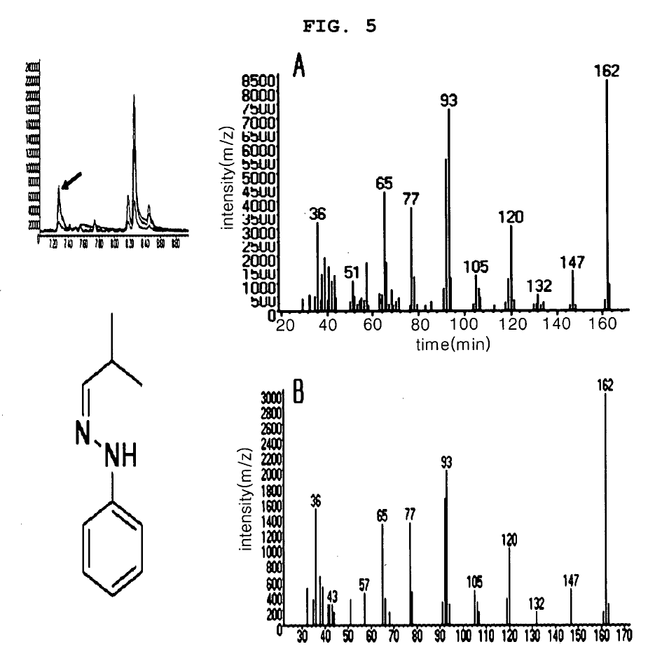

As a result, it was confirmed that the peak at 7.21 min. indicated phenylhydrazone derivative derived from isobutylaldehyde, the peak at 8.18 min. indicated phenylhydrazone derivative derived from 2-methylbutylaldehyde, the peak at 8.26 min. indicated phenylhydrazone derivative derived from isovaleraldehyde, and the peak at 8.44 minute indicated phenylhydrazone derivative derived from valeraldehyde (FIG. 5-FIG. 8).

### EXPERIMENTAL EXAMPLE 3

### Synthesis of Standard Phenylhydrazone Derivative and Comparative Analysis

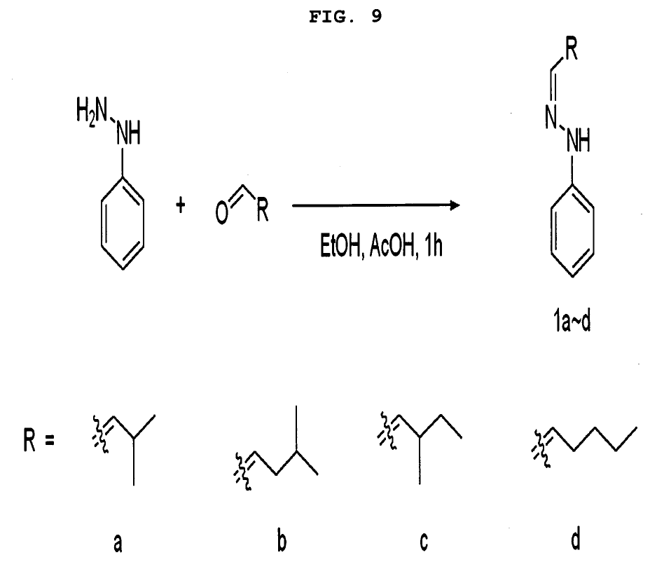

To identify aldehyde, the precursor of phenylhydrazone derivative detected by GC/MSD in Experimental Example <2-2>, corresponding aldehyde was reacted with phenylhydrazine in vitro, resulting in the synthesis of phenylhydrazone derivative. This phenylhydrazone derivative was compared with the phenylhydrazone detected in the urine sample of the irradiated mouse. First, phenylhydrazine (16.5 mM) was reacted respectively with 4 samples (isobutylaldehyde, 2-methylbutylaldehyde, isovaleraldehyde, and valeraldehyde) presumed aldehyde detected in the urine samples in the presence of ethanol solvent supplemented with 1 mM acetic acid for one hour with stirring. Then, 100 ml of distilled water was added to the stirred reaction mixture, followed by extraction with ethyl acetate (200 ml), concentration and purification by flash chromatography (hexane:EtOAc=9:1) using silica gel. As a result, hydrazone derivatives corresponding to each aldehyde were obtained (FIG. 9). The phenylhydrazone derivative synthesized above and the phenylhydrazone detected in the urine sample of the irradiated mouse were analyzed by using GC/MSD respectively and compared.

The phenylhydrazone derivative synthesized by the in-vitro reaction of phenylhydrazine and aldehyde was confirmed to be equal to the phenylhydrazone derivative detected in the urine sample of the irradiated mouse.

1. A screening method of a lipid hydroperoxide regulator comprising the following steps:
1) irradiating the experimental group animals treated with candidate substances and the control group animals;
2) collecting urines from the animals of step 1);
3) performing quantitative and qualitative analysis of lipid hydroperoxide in urines collected in step 2) and comparing the levels between the experimental group and the control group; and
4) selecting a candidate substance that made changes in components or quantity of lipid hydroperoxide by comparing the results of the control group and the experimental group of step 3)

2. The screening method according to claim 1, wherein the test animals of the experimental group and the control group of step 1) are adapted before experiments.

3. The screening method according to claim 1, wherein the candidate substance of step 1) is selected from the group consisting of peptide, protein, non-peptide compound, synthetic compound, fermented product, cell extract, plant extract, animal tissue extract and blood plasma.

4. The screening method according to claim 1, wherein the animal of step 1) is a member of mammals.

5. The screening method according to claim 3, wherein the mammal is selected from the group consisting of mouse, rat, pig and monkey.

6. The screening method according to claim 4, wherein the mouse is selected from the group consisting of Balb.c, ICR and C57BL/6j.

7. The screening method according to claim 4, wherein the rat is SD or Wistar-ST.

8. The screening method according to claim 1, wherein the radiation of step 2) is selected from the group consisting of gamma ray, electron beam, X ray, ion beam and UV.

9. The screening method according to claim 1, wherein the radiation is gamma ray.

10. The screening method according to claim 8, wherein the gamma ray is emitted from a radio-isotope selected from the group consisting of Co-60, Kr-85, Sr-90 and Cs-137.

11. The screening method according to claim 1, wherein the irradiation of step 2) is performed for 3-8 minutes.

12. The screening method according to claim 1, wherein the irradiation is performed at the total absorbed dose of 2-4 Gy.

13. The screening method according to claim 1, wherein the collection of the urine samples of step 2) is performed at 10-15° C.

14. The screening method according to claim 1, wherein the analysis of lipidhydroperoxide of step 3) is performed after synthesizing phenylhydrazone derivative by adding phenylhydrazine to aldehyde detected in the urine sample.

15. The screening method according to claim 13, wherein the phenylhydrazine is selected from the group consisting of 2,4-dinitrophenylhydrazine, 4-chlorophenylhydrazine and 2,4-dichlorophenylhydrazine.

16. A screening method of an anti-oxidant agent comprising the following steps:
1) irradiating the experimental group animals treated with candidate substances and the control group animals;
2) collecting urines from the animals of step 1);
3) performing quantitative and qualitative analysis of lipid hydroperoxide in urines collected in step 2) and comparing the levels between the experimental group and the control group; and
4) selecting a candidate substance capable of inhibiting lipid hydroperoxide by comparing the levels thereof between the control group and the experimental group based on the results of analysis of step 3).
